(19) United States
(12) Patent Application Publication
(10) Pub. No.: US 2007/0032153 A1
(43) Pub. Date: Feb. 8, 2007

(54) Altering the polarity of surfaces with amphipathic peptides

(75) Inventor: William J. Todd, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(73) Assignee: Bd. of Supervisors of Louisiana State University & Agricultural & Mech. College

(21) Appl. No.: 11/198,714

(22) Filed: Aug. 5, 2005

(51) Int. Cl. B32B 27/04; A61K 38/47

(52) U.S. Cl. 442/123; 530/350

(57) ABSTRACT

A method is disclosed for enhancing interactions between nonpolar substances and polar liquids, such as interactions between nonpolar polymers and aqueous systems, using amphipathic peptides, without the need for conventional surfactants. The nonpolar substance may either be pretreated to enhance its interactions with polar liquids, or the polar liquid (e.g., water) may be pretreated to enhance the interactions. It is possible to coat large surface areas on nonpolar substances uniformly with polar liquids. Nonpolar particles may be uniformly suspended in polar liquids, to an extent that has not been previously reported. The alteration of the nonpolar surface can be surprisingly durable. The treated surface may be rinsed, and the treated nonpolar surface will still retain its enhanced properties for interacting with polar liquids. By contrast, conventional surfactants are washed away when the water or other polar liquid is removed.

This invention pertains to compositions and methods for modifying the polarity, wettability, and related properties of otherwise hydrophobic surfaces, in order to enhance their interactions with water, aqueous solutions, or other polar liquids.

There is an unfilled need for improved methods to modify hydrophobic surfaces, for example, the surfaces of hydrophobic polymers, to enhance their interactions with water, aqueous solutions, or other polar liquids. Likewise, there is an unfilled need for improved methods to modify the properties of such polar liquids, so that the liquid will better interact with hydrophobic polymers or other hydrophobic surfaces.

There are many potential applications for such improved methods. They could be used, for example, to enhance the flow of hydrophilic liquids, such as water, over hydrophobic surfaces, and to enhance the evenness of that flow; or to promote the spreading of hydrophilic fluids, e.g. water-based paints, over hydrophobic surfaces and promote binding to the surfaces; or to stably suspend hydrophobic particles in polar liquids, such as in water; or to binding to aggregates of hydrophobic pharmaceuticals to aid their delivery in an aqueous environment.

Prior methods have generally relied on conventional surfactants, such as soaps and detergents, to form emulsions of polar and nonpolar substances. Conventional surfactants typically contain a hydrophilic “head,” such as a carboxyl group, and a hydrophobic “tail,” such as a long alkyl group. In a polar solvent such as water, the surfactant molecules coalesce into spheroids called “micelles,” aggregations in which the hydrophilic groups face outward, and the hydrophobic tails face inward. The interior of the micelle effectively provides a nonpolar solvent to dissolve nonpolar substances that would otherwise be insoluble in a polar solvent. Micelles are dynamic aggregations, continually forming and reforming, exchanging molecules with each other and with the solution phase. Conventional surfactants are subject to a “critical micelle concentration,” a concentration below which, for thermodynamic reasons, the surfactant molecules remain separate in solution, without aggregating into micelles.

Traditional surfactants are subject to various limitations. For example, the dilution of a surfactant below the critical micelle concentration can negate its effectiveness. Also, surfaces treated with traditional surfactants tend to be slippery, and often do not react readily with reagents in aqueous solution or other polar solutions. Furthermore, traditional surfactants have only a limited ability to maintain a stable suspension of hydrophobic particles in aqueous or other polar phase, especially if the particles are large, or if the particles have a high density.

Another approach to enhance the interactions of nonpolar substances is to use hydrophobic solvents. However, most nonpolar solvents are organic compounds that are relatively expensive, at least somewhat toxic, often harmful to the environment, flammable, and otherwise generally more dangerous and more difficult to work with than aqueous systems. In addition, unless the viscosity of an organic liquid is unusually high, it is generally incapable of maintaining stable emulsions of larger particles, including particles formed of nonpolar substances. As with detergents in aqueous systems, larger particles tend to settle out of organic liquids, making it difficult to maintain the uniformity of a suspension.

Another alternative has been to use chemical cross-linking in ultrathin polymer films to promote surface wetting and adhesion between otherwise incompatible fluids and surfaces. See, e.g., D. Ryu et al., “A generalized approach to the modification of solid surfaces,” Science, vol. 308, pp. 236-239 (2005).

So-called “lytic” peptides, or antimicrobial peptides, are low molecular weight peptides that play an important protective role in diverse species, including free-living unicellular organisms, insects, sharks, amphibians, and mammals. A principal function of these peptides is to protect against invading pathogens, such as bacteria. The peptides will lyse the membranes of many types of bacteria, especially Gram-negative bacteria.

Lytic peptides are “amphipathic,” that is, under suitable conditions they tend to assume a conformation in which one side is primarily nonpolar, and the opposite side is primarily polar. In naturally-occurring lytic peptides, the polar side generally bears a positive charge, and the amphipathic conformation is that of an amphipathic alpha helix. An “amphipathic” helix may be depicted as a cylinder in which one face comprises primarily amino acids with hydrophobic (nonpolar) side chains, and in which the opposing face comprises primarily amino acids with charged or polar side chains.

Lytic peptides cause the formation of pores in the bacterial membrane and loss of cellular integrity; membrane blebs that are released from the bacterial membrane; and perhaps even dissolution of the bacterial membrane. One or more of these mechanisms causes bacterial cell death.

Amphipathic lytic peptides are also found in some types of venom, for example melittin from honeybees. The mechanism of action in venoms is essentially similar, based on binding of peptide to the negative surface of cells through electrostatic interactions, through an amphipathic conformation in which a hydrophobic face inserts into the hydrophobic layer of the cell membrane, and thereby disrupts membrane function.

It has been reported that amphipathic peptides can act as surfactants, and that they can have emulsification and foaming activity. M. Enser et al., “De novo design and structure-activity relationships of peptide emulsifiers and foaming agents,” Int. J. Biol. Macromol., vol. 12, pp. 118-124 (1990) discloses the design of eight amphipathic peptides, ranging from 8 to 29 amino acids long; and an investigation into the effects of amino acid composition, peptide length, and secondary structure on surface activity, assessed as emulsification and foaming activity. Emulsification activity for a corn oil/water system was reported to increase rapidly between 11 and 22 amino acid residues as alpha-helicity in aqueous solution increased. The peptides were said to produce stable emulsions as compared with detergents. Foaming activity was enhanced by the presence of aromatic amino acids.

Pulmonary surfactant proteins are essential for normal lung function. Pulmonary surfactant proteins include certain amphipathic, alpha-helical domains. G. Nilsson et al., “Synthetic peptide-containing surfactants,” Eur. J. Biochem., vol. 255, pp. 116-124 (1998) reported, however, that superior fluid flow of lipid mixtures was obtained with an artificial peptide that formed an alpha helix but that lacked amphipathic character; as compared to an artificial peptide formed of the same amino acids, but that formed an amphipathic alpha helix. By improving fluid flow and reducing surface tension, normal and modified surfactants, including proteins, improved the efficiency of oxygen uptake by the lung. The surfactants essentially broke up the thick layers of fluid that can form at the lung surface, for example in premature births.

I have discovered an improved method for enhancing interactions between nonpolar substances and polar liquids, such as interactions between nonpolar polymers and aqueous systems, using amphipathic peptides, without the need for conventional surfactants. The nonpolar substance may either be pretreated to enhance its interactions with polar liquids, or the polar liquid (e.g., water) may be pretreated to enhance the interactions. It is possible to coat large surface areas on nonpolar substances uniformly with polar liquids. Nonpolar particles may be uniformly suspended in polar liquids, to an extent that has not been previously reported.

The alteration of the nonpolar surface can be surprisingly durable. E.g., unlike a surface treated with a conventional surfactant, after the novel treatment the liquid phase may be removed entirely, the treated surface may be rinsed, and the treated nonpolar surface will still retain its enhanced properties for interacting with polar liquids. By contrast, conventional surfactants are washed away when the water or other polar liquid is removed; or, even if trace amounts of surfactant may remain on the surface of the hydrophobic substance, their concentration will be below the critical micelle concentration of the surfactant if water or other polar liquid is later added. The invention may be used with virtually any shape of particle or surface. It may be employed with a wide variety of hydrophobic substances and polar liquids.

The invention may be practiced with almost any hydrophobic polymer capable of forming a surface or particle. Such compounds include, by way of example and not limitation, the following polymer families and their derivatives: acrylates, polyalkyls, polyolefins, polydienes, polyacetones, polylactides, polysiloxanes, polyoxiranes, polystyrenes, polyethylenes, polypropylenes, fluorinated ethylene propylenes such as polytetrafluoroethylene, silicone polymers, and the like.

The responses of different classes of different hydrophobic polymers to the same amphipathic peptide can vary. For example, when 10 μL of a 1 mg/mL aqueous melittin solution was applied to a polystyrene surface, the small drop could easily be spread out to cover an area at least two centimeters in diameter; whereas on a polytetrafluoroethylene surface an equivalent drop could only be spread over an area up to about one centimeter in diameter, and more effort was required to produce uniform spreading.

The structure of the peptide may vary to optimize results, for example by increasing the length and hydrophobic character of the hydrophobic face to enhance binding to and modification of extremely hydrophobic polymers such as polytetrafluoroethylene (Teflon™).

Peptides capable of forming amphipathic helices have characteristics that are distinct from detergents or conventional surfactants. The primary structure of the peptide, the linear sequence of its amino acids, does not reveal a clear separation into hydrophobic and hydrophilic domains; rather, the primary structure is a mixture of both hydrophobic and hydrophilic amino acids. By contrast, the primary structure of a conventional surfactant has clearly divided hydrophobic and hydrophilic domains. However, an amphipathic peptide is capable of assuming a secondary structure in which the side chains move into position to create an amphipathic conformation, with one side of the alpha helix predominately hydrophobic and the opposite side predominately hydrophilic. In aqueous solutions the peptides often adopt a near-random conformation, rather than form the micelles that are characteristic of detergents. However, as the hydrophobicity of the solvent increases, or as the molecules contact hydrophobic domains or surfaces, their steric orientation can rearrange into an amphipathic alpha helical conformation as being thermodynamically more compatible with that environment. The hydrophobic side groups associate with the hydrophobic structure, while the hydrophilic side groups on the opposite face interact with the polar solvent (e.g., water).

The amphipathic peptides in this invention do not behave as conventional surfactants. They do not (or at least need not) form micelles. They can remain bound to the nonpolar surface upon rinsing. They do not have a critical micelle concentration, although their efficacy does increase, to a point, with increasing concentration.

The polar side groups may be uncharged, positively charged, negatively charged, or a mixture of these three possibilities. Which is selected can vary, depending on the particular application. For example, for suspending hydrophobic particles in aqueous solutions like charges are generally preferred, because like charges cause the particles to repel one another, thereby promoting the formation of uniform suspensions and stable colloids. By contrast, in coating hydrophobic surfaces the charge could be positive, negative, or mixed.

Naturally occurring amphipathic peptides are known for their ability to disrupt membrane function. For this reason they are often called lytic or antimicrobial peptides. In naturally occurring lytic peptides, positive side groups predominate in the amino acids on the hydrophilic face. That face is positively-charged to promote interaction with the predominately negatively surface charge of targeted cell membranes. In general, the present invention will work whether the charges are positive or negative. Negatively charged groups may predominate if, for example, the peptide is made by biological processes, or if the intended uses involve exposure to cells or tissues that should not be lysed.

The design of peptides with the characteristics of an amphipathic alpha helix is relatively straightforward, and may be facilitated, for example by using the well known helical wheel as an aid to determine appropriate positioning of amino acids. The “traditional” amphipathic helix structure may be modified, for example by N- or C-terminal additions and modifications, provided that amphipathic character is retained in at least part of the peptide. Additional sequences of amino acids may generally be added to the C- or N-terminal regions or both, without altering the ability of the amphipathic helical portion of the molecule to bind to and alter the polarity of surfaces. Such additions may be desirable, for example, to help target the molecule to specific sites or receptors—for example, as an aid to facilitated transport across biological barriers, to bring the complex in contact with cells possessing specific receptors, or to direct the complex to specific sites on solid surfaces. As examples of amphipathic peptides with such leader sequences, conjugates of both Hecate and Phorl4 were separately linked to the receptor binding sequence of luteinizing hormone. (See, e.g., W. Hansel et al., “Targeted destruction of prostate cancer cells and xenografts by lytic peptide-βLH conjugates,” Reprod. Biol. vol. 1, pp. 20-32 (2001).) Both conjugates were found to be effective, at a concentration of 1 mg/ml in PBS, to allow hydrophobic C₁₈ particles to enter and remain suspended in aqueous solution.

The length of the amphipathic alpha helix may vary; longer peptides can be more effective for some applications. In general, it is preferred that the amphipathic portion of the peptide be 14 amino acids or longer, more preferably between about 20and about 40 amino acids. Longer peptides will, in general, bind more strongly to hydrophobic surfaces than otherwise similar but shorter peptides. With extremely hydrophobic surfaces, longer peptides are particularly preferred. A peptide in an amphipathic alpha helix has flexibility, which allows firm binding over variations in the contours that may exist in the hydrophobic surface at the molecular level.

The nature both of the charged or polar amino acids, and of the nonpolar amino acids may be varied as desired, provided that amphipathic character is maintained. The identity of the specific amino acids may be chosen, for example, to promote subsequent reactions, such as crosslinking other molecules to the hydrophobic surface, or otherwise to promote desired interactions with constituents in the aqueous or other polar phase solution.

The present invention is not limited to peptides containing solely the “standard” 20 amino acids, although it will often be convenient to use those 20 amino acids, especially if the peptides are to be synthesized biologically, e.g., in E. coli. Of the “standard” 20 amino acids, the side chains of the following are hydrophobic: Ala, Val, Leu, IIe, Pro, Trp, Phe, and Met. The side chains of the following amino acids are uncharged but polar: Gly, Ser, Thr, Tyr, Cys, Asn, and Gln. The side chains of the following amino acids are basic (and therefore positively charged at neutral pH): Lys, Arg, and His. The side chains of the following amino acids are acidic (and therefore negatively charged at neutral pH): Asp, Glu.

Numerous variations in the amino acid sequences of the peptides that may be used in this invention are possible, provided that the amino acid sequence of the peptide assumes an amphipathic alpha helical conformation in the presence of a polar liquid and a nonpolar surface. Numerous examples may readily be designed using a “helical wheel” to determine the relative positions of hydrophilic and hydrophobic amino acids along the length of the alpha helix, with an average of about 3.5 amino acids per helical turn. It is preferred that the peptide should be about 14 amino acid residues or longer, or about 4 helical turns, more preferably about 21-28 amino acids. There is no upper size limitation on the peptides or proteins that may be used in the invention, aside from the practicalities of producing larger peptides or proteins. To interact with the polar liquid, polarity in the amino acid side group is all that is required, although full charges are generally more effective. On the hydrophobic face, longer chain and ring structures on non-polar side groups are preferred.

Peptides for use in the present invention may be produced through any of a variety of methods known in the art for preparing peptides, including, for example, production in a peptide synthesizer, or production in a biological system containing a nucleic acid encoding the desired peptide or a precursor. Such biological systems include, for example, transformed E. coil; S. cerevisiae; green plants; transformed goats, cows, or rabbits that secrete exogenous peptide in milk; and transformed chickens that secrete exogenous peptide in egg white.

For initial comparisons intended primarily to identify preferred peptides for particular applications, in most instances it will be faster to prepare peptides by synthetic chemical means. Once an optimized or desired peptide sequence has thus been selected, a biological production system will often be preferred as being more cost-effective for the large-scale production of peptide. This “biological” approach to modifying surfaces may be scaled up more economically than other means that have been used for modification of solid surfaces.

Optionally, the peptides may be cross-linked after binding to the hydrophobic surface, thus enhancing the long-term stability of the surface modification. If the structure to be modified is a small particle or a long thin thread, the peptides may be linked completely around the structure, forming a kind of molecular net and increasing stability. Alternatively, or in addition, having cross-linkable groups on the peptide can facilitate the attachment of dyes, ligands, biotin, avidin, or other molecules of interest. The amphipathic peptide may thus act as a bridge or linker between the hydrophobic surface and molecules that will not ordinarily react directly with a hydrophobic surface. Covalent cross-linking of peptides on the polymer surface may be conducted using techniques and linkers known in the art, such as disulfide bonds, glutaraldehyde, amide linkages, bis(imido esters), diisocyanates, dibromobimane, an amine-thiol heterobifunctional crosslinker such as one containing both an succinimidyl ester and a maleimide, or an amine-carboxylic acid crosslink via a carbodiimide.

Previously, the biological production of lytic or antimicrobial peptides has been problematic, because such peptides tend to be lethal to the cells that produce the peptides. Amphipathic, alpha helical peptides having a positively charged polar face are generally toxic. The positively-charged face interacts with negatively-charged surface of the membrane of the cell that expresses the lytic peptides, thus killing the cell as otherwise described above. To circumvent such problems, lytic peptides have sometimes been expressed as a propeptide. i.e., a lytic domain is fused to a blocking domain that may be cleaved after expression to generate the active form of the lytic peptide. While the propeptide approach may certainly be used in practicing this invention, it may not be necessary to add that layer of complexity. In practicing this invention, it is not required that the polar face of the alpha helical domain must contain positively-charged amino acids. Instead, that face may contain, positive, negative, or polar amino acid residues, or a mixture of such residues, provided that the resulting polar face has sufficient polarity to interact with water or other polar liquids. Production of such peptides by cloning technologies is simplified and does not require the use of a propeptide intermediate. The expressed amphipathic peptide may be effective for the purposes of this invention, and yet be non-toxic.

In a simple aqueous solution the amphipathic structure does not form; rather the stable conformation is apparently an entangled structure. There is no thermodynamic drive towards an amphipathic conformation. In nature the rearrangementto form a functional amphipathic conformation is apparently triggered by interactions between positively charged side groups on the peptide and negative charges on the cell membrane (e.g., on phospholipids, sugars, and perhaps some proteins). The charge interaction alters the conformation of the peptide, forming a hydrophobic face and a hydrophilic face on opposite sides of the alpha helix. The thermodynamic interaction between the hydrophobic domain and the water drives the hydrophobic portion of the amphipathic structure into the cell membrane. When a sufficient number of amphipathic peptides are incorporated into the membrane, the cell lyses, possibly due to the peptide molecules' formation of pores in the membrane. By contrast, if the side-chains on the peptide were negative, the structure would tend to be repelled from the surface of the negatively charged membrane.

The invention may optionally be used to improve bonding between hydrophobic and hydrophilic materials when one is applied in a liquid state. For example, a hydrophilic paint adheres betterto a hydrophobic polymer surface when the surface is first bonded to amphipathic peptides in accordance with the present invention.

While aqueous solutions of amphipathic peptides have certainly come into contact with polymeric surfaces in the past, to the knowledge of the inventor it has never been suggested that the hydrophilicity of the polymeric surface might change as a result. To the knowledge of the inventor, any such changes in surface properties have not previously been appreciated. Polymeric materials have previously been used to contain, transport, measure, or purify substances containing amphipathic peptides, such as aqueous solutions of those peptides. Thus, one aspect of this invention is an article of manufacture comprising a nonpolar polymer and an amphipathic peptide on at least a portion of the surface of the polymer, wherein the article is not adapted to contain, transport, measure, or purify a substance. For example, such an article might be a fiber, thread, yarn, or fabric.

### EXAMPLE 1

Melittin, extracted from honeybee venom, was used as the amphipathic peptide in several proof-of-concept experiments, because this peptide is relatively inexpensive to purchase.

Water normally beads up unevenly on polystyrene surfaces, and does not readily “wet” those surfaces. However, when melittin was first dissolved in water (1 mg/mL), the water was easily spread evenly over polystyrene surfaces. If the aqueous melittin solution was removed from the polystyrene, and the polystyrene permitted to dry, the polymer retained its wettability when later exposed to deionized water, which again spread evenly over the polystyrene surface. The amphipathic peptide modifications to the polystyrene were thus retained after removal of the water and drying in air.

Attempts to physically induce water to spread on a polystyrene surface, for example by using a glass hockey-stick shaped instrument to force a water droplet across the surface, invariably failed: the droplet remained intact and did not flatten out or otherwise “wet” the surface. However, when melittin was first dissolved in water (1 mg/ml), the drop readily spread across the polystyrene surface to form a uniform thin film. The aqueous melittin solution was then washed from the polystyrene using tap water, and the polystyrene was allowed to dry in air. After drying, the treated polystyrene retained the ability to be wetted by water, and to allow water to spread across the treated surface in a thin film. In an alternative, the polystyrene surface with the spread melittin solution was baked to dryness at 60° C, and rinsed in tap water. This polystyrene also retained the ability to allow water to spread across the surface.

Forcomparison, drops of aqueous solutions of 1% SDS and 1% Triton X-100 were applied in the same manner. These drops readily spread across the polystyrene surface. However both samples lost the ability to be “wetted” by water after rinsing in tap water, even when the sample was first baked to dryness. The binding affinity and stability of the amphipathic peptide to the hydrophobic surface were much stronger than those of the detergents, although both types of molecules had been equally effective in promoting the initial spread of water across the hydrophobic surface.

### EXAMPLE 2

Fabric formed from polypropylene fibers forms a lightweight, comfortable textile having good insulation properties. However, such a fabric is normally hydrophobic, and does not readily wick water, making it unsuitable for many textile applications. A commercially-obtained, hydrophobic, woven polypropylene cloth was converted into a hydrophilic fabric, one that readily wicked water, simply by dipping it in an aqueous solution of melittin (1 mg/mL). The treated polypropylene cloth retained its wettability even after several cycles of rinsing and air-drying.

The treated material possessed sufficient wicking to transport water up and overthe edge of a beaker. By contrast, the original, untreated polypropylene material remained incompatible with the water. Such a treated material could, for example, be used as a fabric for sporting activities, maintaining warmth while wicking sweat away from the skin. If the wicking action should become less effective over time, it could be restored by again rising the material in a solution containing the amphipathic peptide. By contrast, when the same polypropylene material was immersed in 1% SDS or 1% Triton X-100 detergent solution, the polypropylene fabric mixed with the water; however this characteristic was only transient—rinsing in tap water promptly remove the detergents, and the fabric readily regained its hydrophobic character.

### EXAMPLE 3

Hydrophobic C₁₈ particles, approximately 60 to 100 μm in diameter, of the type commonly used in chromatographic separation columns, are not normally miscible with water. However, after treatment with a solution of melittin (1 mg/mL), the formerly hydrophobic C₁₈ particles were readily suspended in water. The particles were then repeatedly dried and successfully re-suspended in deionized water.

### EXAMPLE 4

Detergents prevent sticking or binding of substances to a surface by creating a slippery coating of micelles across the surface. Detergents are also commonly used to dislodge and remove attached substances from surfaces. By contrast, amphipathic peptides may be used in accordance with the present invention to improve the binding properties of hydrophobic surfaces, such as to promote the attachment and growth of cells in Petri dishes, or to coat microtiterwells to promote attachment of antigens.

Detergents are highly effective in altering the properties of water to promote intimate contact with hydrophobic surfaces. This property was tested using four common detergents: sodium dodecyl sulfate (SDS), Triton X-100, Nonidet P-40, and Tween 20, each at a concentration of 1 mg/ml in phosphate buffered saline (PBS). The detergent solutions were tested for their ability to take up hydrophobic C₁₈ particles, such as are commonly used in separation column chromatography. The C₁₈ particles ranged from about 60 to about 100 micrometers in diameter. In unmodified PBS, the C₁₈ particles floated on the top surface, and did not penetrate the fluid. By contrast, each of the detergents caused the particles readily to enter the detergent-containing PBS; however, they did not remain suspended and in all cases soon settled to the bottom of the tube.

By contrast, when PBS was mixed with 1 mg/ml of the amphipathic peptide melittin, the C₁₈ particles not only entered the fluid, but they remained suspended over a test period of several weeks and did not settle out. The melittin-PBS system held up to about 0.5 mg of C₁₈ particles per milliliter of solution. Above this level additional C₁₈ particles remained on the surface of the fluid, presumably because the free melittin had all been bound to C₁₈ at that point.

The properties of traditional surfactants and of the amphipathic peptides were different. These observations suggest that where the goal is to maximize the amount of hydrophobic material that enters a hydrophilic fluid, then more traditional surfactants may be the agent of choice; however, where the goal is the long-term, stable suspension of hydrophobic particles in a hydrophilic fluid, then amphipathic peptides may be preferred.

### Lytic Peptides Useful in the Present Invention

Many lytic peptides are known in the art and include, for example, those mentioned in the references cited in the following discussion.

Lytic peptides are small, amphipathic peptides. Native lytic peptides appear to be major components of the antimicrobial defense systems of a number of animal species, including those of insects, amphibians, and mammals. They typically comprise 23-39 amino acids, although they can be smaller. They have the potential for forming amphipathic alpha-helices. Naturally-occurring lytic peptides are nearly always basic. See Boman et al., “Humoral immunity in Cecropia pupae,” Curr. Top. Microbiol. Immunol. vol. 94/95, pp. 75-91 (1981); Boman etal., “Cell-free immunity in insects,” Annu. Rev. Microbiol., vol. 41, pp. 103-126 (1987); Zasloff, “Magainins, a class of antimicrobial peptides from Xenopus skin: isolation, characterization of two active forms, and partial DNA sequence of a precursor,” Proc. Natl. Acad. Sci. USA, vol. 84, pp. 3628-3632 (1987); and Lee et al., “Antibacterial peptides from pig intestine: isolation of a mammalian cecropin,” Proc. Natl. Acad. Sci. USA, vol. 86, pp. 9159-9162 (1989).

Known amino acid sequences for lytic or amphipathic peptides may be modified to create new peptides by substitutions of amino acid residues that preserve the amphipathic nature of the peptides (e.g., replacing a polar or charged residue with another polar or charged residue, or a non-polar residue with another non-polar residue, etc.); or by lengthening or shortening the amino acid sequence while preserving its amphipathic character or its charge distribution. Note that, for use in the present invention, it is necessary only to preserve the amphipathic nature of the peptide, but not necessarily lytic or antimicrobial properties; thus polar residues may be substituted for charged residues, or vice versa; and acidic residues may be substituted for basic residues, or vice versa.

Lytic peptides and their sequences are disclosed in Yamada et al., “Production of recombinant sarcotoxin IA in Bombyx mori cells,” Biochem. J., vol. 272, pp. 633-666 (1990); Taniai et al., “Isolation and nucleotide sequence of cecropin B cDNA clones from the silkworm, Bombyx mori,” Biochimica Et Biophysica Acta, vol. 1132, pp. 203-206 (1992); Boman et al., “Antibacterial and antimalarial properties of peptides that are cecropin-melittin hybrids,” Febs Letters, vol. 259, pp. 103-106 (1989); Tessier et al., “Enhanced secretion from insect cells of a foreign protein fused to the honeybee melittin signal peptide,” Gene, vol. 98, pp. 177-183 (1991); Blondelle et al., “Hemolytic and antimicrobial activities of the twenty-four individual omission analogs of melittin,” Biochemistry, vol. 30, pp. 4671-4678 (1991); Andreu et al., “Shortened cecropin A-melittin hybrids. Significant size reduction retains potent antibiotic activity,” Febs Letters, vol. 296, pp. 190-194 (1992); Macias et al., “Bactericidal activity of magainin 2: use of lipopolysaccharide mutants,” Can. J. Microbiol., vol. 36, pp. 582-584 (1990); Rana et al., “Interactions between magainin-2 and Salmonella typhimurium outer membranes: effect of Lipopolysaccharide structure,” Biochemistry, vol. 30, pp. 5858-5866 (1991); Diamond et al., “Airway epithelial cells are the site of expression of a mammalian antimicrobial peptide gene,” Proc. Natl. Acad. Sci. USA, vol. 90, pp. 4596 (1993); Lehrer et al., Blood, vol. 76, pp. 2169-2181 (1990); Ganz et al., Sem. Resp. Infect. I., pp. 107-117 (1986); Kagan et al., Proc. Natl. Acad. Sci. USA, vol. 87, pp. 210-214 (1990); Wade et al., Proc. Natl. Acad. Sci. USA, vol. 87, pp. 4761-4765 (1990); Romeo et al., J. Biol. Chem., vol. 263, pp. 9573-9575 (1988); Jaynes et al., “Therapeutic Antimicrobial Polypeptides, Their Use and Methods for Preparation,” WO 89/00199 (1989); Jaynes, “Lytic Peptides, Use for Growth, Infection and Cancer,” WO 90/12866 (1990); Berkowitz, “Prophylaxis and Treatment of Adverse Oral Conditions with Biologically Active Peptides,” WO 93/01723 (1993).

Families of naturally-occurring lytic peptides include the cecropins, the sarcotoxins, the melittins, and the magainins. Boman and coworkers in Sweden performed the original work on the humoral defense system of Hyalophora cecropia, the giant silk moth, to protect itself from bacterial infection. See Hultmark et al., “Insect immunity. Purification of three inducible bactericidal proteins from hemolymph of immunized pupae of Hyalophora cecropia,” Eur. J. Biochem., vol. 106, pp. 7-16 (1980); and Hultmark et al., “Insect immunity. Isolation and structure of cecropin D. and four minor antibacterial components from cecropia pupae,” Eur. J. Biochem., vol. 127, pp. 207-217 (1982).

Infection in H. cecropia induces the synthesis of specialized proteins capable of disrupting bacterial cell membranes, resulting in lysis and cell death. Among these specialized proteins are those known collectively as cecropins. The principal cecropins—cecropin A, cecropin B, and cecropin D—are small, highly homologous, basic peptides. In collaboration with Merrifield, Boman's group showed that the amino-terminal half of the various cecropins contains a sequence that will form an amphipathic alpha-helix. Andrequ et al., “N-terminal analogues of cecropin A: synthesis, antibacterial activity, and conformational properties,” Biochem., vol. 24, pp. 1683-1688 (1985). The carboxy-terminal half of the peptide comprises a hydrophobic tail. See also Boman et al., “Cell-free immunity in Cecropia,” Eur. J. Biochem., vol. 201, pp. 23-31 (1991).

A cecropin-like peptide has been isolated from porcine intestine. Lee et al., “Antibacterial peptides from pig intestine: isolation of a mammalian cecropin,” Proc. Natl. Acad. Sci. USA, vol. 86, pp. 9159-9162 (1989).

Cecropin peptides have been observed to kill a number of animal pathogens other than bacteria. See Jaynes et al., “In Vitro Cytocidal Effect of Novel Lytic Peptides on Plasmodium falciparum and Trypanosoma cruzi,” FASEB, 2878-2883 (1988); Arrowood et al., “Hemolytic properties of lytic peptides active against the sporozoites of Cryptosporidium parvum,” J. ProtozooL, vol. 38, No. 6, pp. 161S-163S (1991); and Arrowood et al., “In vitro activities of lytic peptides against the sporozoites of Cryptosporidium parvum,” Antimicrob. Agents Chemother., vol. 35, pp. 224-227 (1991). However, normal mammalian cells do not appear to be adversely affected by cecropins, even at high concentrations. See Jaynes et al., “In vitro effect of lytic peptides on normal and transformed mammalian cell lines,” Peptide Research, vol. 2, No. 2, pp. 1-5 (1989); and Reed et al., “Enhanced in vitro growth of murine fibroblast cells and preimplantation embryos cultured in medium supplemented with an amphipathic peptide,” Mol. Reprod. Devel., vol. 31, No. 2, pp. 106-113 (1992).

Slightly larger peptides called sarcotoxins have been purified from the fleshfly Sarcophaga peregrina. Okada et al., “Primary structure of sarcotoxin l, an antibacterial protein induced in the hemolymph of Sarcophaga peregrina (flesh fly) larvae,” J. Biol. Chem., vol. 260, pp. 7174-7177 (1985). Although highly divergent from the cecropins, the sarcotoxins presumably have a similar antibiotic function.

Other lytic peptides have been found in amphibians. Gibson and collaborators isolated two peptides from the African clawed frog, Xenopus laevis, peptides which they named PGS and Gly¹⁰Lys²²PGS. Gibson et al., “Novel peptide fragments originating from PGLₐ and the caervlein and xenopsin precursors from Xenopus laevis,” J. Biol. Chem., vol. 261, pp. 5341-5349 (1986); and Givannini et al., “Biosynthesis and degradation of peptides derived from Xenopus laevis prohormones,” Biochem. J., vol. 243, pp. 113-120 (1987). Zasloff showed that the Xenopus-derived peptides have antimicrobial activity, and renamed them magainins. Zasloff, “Magainins, a class of antimicrobial peptides from Xenopus skin: isolation, characterization of two active forms, and partial DNA sequence of a precursor,” Proc. Natl. Acad. Sci. USA, vol. 84, pp. 3628-3632 (1987).

Synthesis of nonhomologous analogs of different classes of lytic peptides has been reported to reveal that a positively charged, amphipathic sequence containing at least 20 amino acids appeared to be a requirement for lytic activity in some classes of peptides. Shiba et al., “Structure-activity relationship of Lepidopteran, a self-defense peptide of Bombyx more,” Tetrahedron, vol. 44, No. 3, pp. 787-803 (1988). Other work has shown that smaller peptides can also be lytic. See McLaughlin et al., cited below.

Cecropins have been shown to target pathogens or compromised cells selectively, without affecting normal host cells. The synthetic lytic peptide known as S-1 (or Shiva 1) has been shown to destroy intracellular Brucella abortus-, Trypanosoma cruzi-, Cryptosporidium parvum-, and infectious bovine herpes virus I (IBR)-infected host cells, with little or no toxic effects on noninfected mammalian cells. See Jaynes et al., “In vitro effect of lytic peptides on normal and transformed mammalian cell lines,” Peptide Research, vol. 2, No. 2, pp. 1-5 (1989); Wood et al., “Toxicity of a Novel Antimicrobial Agent to Cattle and Hamster cells In vitro,” Proc. Ann. Amer. Soc. Anim. Sci., Utah State University, Logan, UT. J. Anim. Sci. (Suppl. 1), vol. 65, p. 380 (1987); Arrowood et al., “Hemolytic properties of lytic peptides active against the sporozoites of Cryptosporidium parvum,” J. Protozool., vol. 38, No. 6, pp. 161S-163S (1991); Arrowood et al., “In vitro activities of lytic peptides against the sporozoites of Cryptosporidium parvum,” Antimicrob. Agents Chemother., vol. 35, pp. 224-227 (1991); and Reed et al., “Enhanced in vitro growth of murine fibroblast cells and preimplantation embryos cultured in medium supplemented with an amphipathic peptide,” Mol. Reprod. Devel., vol. 31, No. 2, pp. 106-113 (1992).

Morvan et al., “In vitro activity of the antimicrobial peptide magainin 1 against Bonamia ostreae, the intrahemocytic parasite of the flat oyster Ostrea edulis,” Mol. Mar. Biol., vol. 3, pp. 327-333 (1994) reports the in vitro use of a magainin to selectively reduce the viability of the parasite Bonamia ostreae at doses that did not affect cells of the flat oyster Ostrea edulis.

Also of interest are the synthetic peptides disclosed in the following pending patent applications, peptides that have lytic activity with as few as 10-14 amino acid residues: McLaughlin et al., U.S. Pat. Nos. 5,789,542 and 6,566,334. The Phor peptides, typified by Phor14 and Phor21, may also be used in practicing this invention. See, e.g., Hansel et al. (2001).

The complete disclosures of all references cited in this specification are hereby incorporated by reference. In the event of an otherwise irreconcilable conflict, however, the present specification shall control.

What is claimed:

1. An article of manufacture comprising a nonpolar polymer and an amphipathic, alpha-helical peptide; wherein at least a portion of the surface of said polymer is bound to said peptide; wherein that portion of the surface to which said peptide is bound has an enhanced ability to interact with polar liquids as compared to the surface of an otherwise identical polymer lacking said peptide on its surface; and wherein said article of manufacture is not adapted to contain, to transport, to measure, or to purify a substance.

2. An article of manufacture as recited in claim 1, wherein said peptide comprises a ligand domain having affinity for a receptor molecule.

3. An article of manufacture as recited in claim 1, wherein said peptide molecules are cross-linked to one another on the surface of said polymer.

4. An article of manufacture as recited in claim 1, wherein said peptide molecules are bound to other molecules.

5. An article of manufacture as recited in claim 1, wherein said article of manufacture comprises a fiber, a thread, a yarn, or a fabric; and wherein said article of manufacture has a substantially greater ability to wick polar liquids than does an otherwise identical article of manufacture that lacks said bound peptides.

6. An article of manufacture as recited in claim 1, wherein said article of manufacture is adapted to retain most of said peptide bound to the surface of said polymer if said article of manufacture is rinsed with a polar liquid, and thereafter to retain its enhanced ability to interact with polar liquids following such rinsing.

7. An article of manufacture comprising a nonpolar polymer and an amphipathic, alpha-helical peptide; wherein at least a portion of the surface of said polymer is bound to said peptide; wherein at least a portion of said peptide molecules are cross-linked to one another; and wherein that portion of the surface to which said peptide is bound has an enhanced ability to interact with polar liquids as compared to the surface of an otherwise identical polymer lacking said peptide on its surface.

8. An article of manufacture as recited in claim 7, wherein said peptide comprises a ligand domain having affinity for a receptor molecule.

9. An article of manufacture as recited in claim 7, wherein said article of manufacture comprises a fiber, a thread, a yarn, or a fabric; and wherein said article of manufacture has a substantially greater ability to wick polar liquids than does an otherwise identical article of manufacture that lacks said bound peptides.

10. An article of manufacture as recited in claim 7, wherein said article of manufacture is adapted to retain most of said peptide bound to the surface of said polymer if said article of manufacture is rinsed with a polar liquid, and thereafter to retain its enhanced ability to interact with polar liquids following such rinsing.

11. A process for enhancing the ability of a nonpolar polymer to interact with a polar liquid; said process comprising binding at least a portion of the surface of the polymerto an amphipathic, alpha helical peptide; wherebythat portion of the surface to which the peptide is bound has an enhanced ability to interact with polar liquids as compared to the surface of an otherwise identical polymer lacking the peptide on its surface.

12. A process as recited in claim 11, wherein the polymer is not an article of manufacture adapted to contain, to transport, to measure, or to purify a substance.

13. A process as recited in claim 11, wherein the peptide comprises a ligand domain having affinity for a receptor molecule.

14. A process as recited in claim 11, additionally comprising the step of cross-linking the peptide molecules to one another on the surface of the polymer.

15. A process as recited in claim 11, additionally comprising the step of binding the peptide molecules to other molecules.

16. A process as recited in claim 11, wherein the polymer comprises a fiber, a thread, a yarn, or a fabric; and wherein the fiber, thread, yard, or fabric has a substantially greater ability to wick polar liquids than does an otherwise identical fiber, thread, yard, or fabric that lacks the bound peptides.

17. A process as recited in claim 11, additionally comprising the step of rinsing the surface of the polymer with a polar liquid lacking any substantial concentration of any amphipathic, alpha helical peptide; wherein that portion of the surface of the polymerto which the amphipathic, alpha helical peptide had previously been bound remains bound to most of the previously-bound peptide; whereby the surface to which the peptide is bound retains its enhanced ability to interact with polar liquids following said rinsing.

18. A process as recited in claim 11, wherein the polymer comprises a plurality of particles; and wherein said process additionally comprises the step of suspending the particles in an aqueous system; wherein following said suspending step the particles remain suspended in the aqueous system for an extended period of time, under conditions for which otherwise identical polymer particles that lack the bound amphipathic peptides would settle from the aqueous system substantially more rapidly.

19. A process as recited in claim 18, wherein the particles remain suspended for at least 24 hours in the absence of stirring.

20. A process as recited in claim 18, wherein the particles remain suspended for at least 7 days in the absence of stirring.

21. A process as recited in claim 18, wherein the suspended particles in the aqueous system form a colloid.

22. The suspension produced by the process of claim 18.

23. A process as recited in claim 11, additionally comprising the step of uniformly spreading a polar liquid over the surface of the peptide-bound polymer.

24. A process as recited in claim 11, additionally comprising the step of bonding to the surface of the polymer a hydrophilic material applied in the liquid state, wherein the hydrophilic material bonds substantially more strongly than an identical hydrophilic material would bond to an otherwise identical nonpolar polymer surface that lacks the amphipathic peptides.
